(19) United States
(12) Patent Application Publication
(10) Pub. No.: US 2009/0252804 A1
(43) Pub. Date: Oct. 8, 2009

(54) MEDICAL DEVICES WITH AN ANTIBACTERIAL POLYURETHANEUREA COATING

(75) Inventors: Juergen Koecher, Langenfeld (DE); Stefanie Eiden, Leverkusen (DE); Anke Mayer-Bartschmid, Wuelfrath (DE); Igor Knezevic, Duesseldorf (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/419,597

(22) Filed: Apr. 7, 2009

(30) Foreign Application Priority Data
Apr. 8, 2008 (EP) 08154208.6

(51) Int. Cl. A61K 9/14; A01N 59/16; A61K 31/74

(52) U.S. Cl. 424/489; 424/618; 424/78.08; 977/773

(57) ABSTRACT

The present invention relates to a medical device which has at least one coating which is obtained starting from an aqueous dispersion, the aqueous dispersion comprising at least one nonionically stabilized polyurethaneurea and at least one silver-containing constituent.

### RELATED APPLICATIONS

This application claims benefit to European Patent Application No. 08 154 208.6, filed Apr. 8, 2008, which is incorporated herein by reference in its entirety for all useful purposes.

### BACKGROUND OF THE INVENTION

The present invention relates to medical devices having an antibacterial (antimicrobial) polyurethaneurea coating. Further provided by the present invention is a process for producing the medical devices with an antibacterial (antimicrobial) polyurethane coating, and also their use.

Articles made of plastic and metal are used very frequently in the medical sector. Examples of such materials are, for example, implants, cannulas or catheters. A problem associated with the use of these products is the ease with which the surfaces of these materials can be colonized by microbes. The consequences of using an article colonized with bacteria, such as an implant, a cannula or a catheter, are often infections through the formation of a biofilm. Such infections are particularly serious in the area of central venous catheters and also in the urological area, where catheters are used.

Before now, in the past, numerous attempts have been made to prevent the colonization of surfaces by bacteria, and hence infections. Often it has been attempted to impregnate the surface of medical implants or catheters with antibiotics. In that case, however, the development and selection of resistant bacteria must be expected.

Another approach to preventing infections when using implants or catheters is to use metals or metal alloys, in the case of catheters, for example.

Of particular significance in this context is the antibacterial effect of silver. Silver and silver salts have already been known for many years to be antimicrobially active substances. The antimicrobial effect of surfaces which contain silver derives from the release of silver ions. The advantage of silver consists in its high toxicity for bacteria, even at very low concentrations. Hardes et al., Biomaterials 28 (2007) 2869-2875, report bactericidal activity for silver at a concentration of down to 35 ppb. In contrast, even at a significantly higher concentration, silver is still not toxic to mammalian cells. A further advantage is the low tendency of bacteria to develop resistances to silver.

Various approaches at equipping medical devices with silver, such as catheters, for example, are described in the literature. One approach is the use of metallic silver on catheter surfaces. Thus U.S. Pat. No. 3,800,087, for example, discloses a process for metallizing surfaces, which according to DE 43 28 999 can also be used for medical devices, such as a catheter, for example. A disadvantage there is that the silver adheres poorly in the face of the challenges on the catheter, such as, for example, on storage in body fluids such as urine, on friction during introduction and removal from the body, or by repeated bending of the catheter.

Metallic coatings on medical devices, however, not only have the disadvantage of the poor adhesion to the catheter material but also the fact that application to the insides of the catheter is very involved at the least.

An improvement to the adhesion of the silver coat on a catheter plastic is described by the aforementioned DE 43 28 999 A, by the application between plastic and silver coat of metal layers with better adhesion. In the case of the products described, the silver is applied by vapour deposition in a vacuum chamber, by sputtering or else by ion implantation. These processes are very complex and costly. A further disadvantage is that the amount of elemental silver applied by vapour deposition is relatively high, while only very small amounts of active silver ions are delivered to the surrounding fluid. Furthermore, these processes can only be used to coat the outside of an implant or a catheter. It is known, however, that bacteria also attach readily to the inside of a catheter, leading to the formation of a biofilm and the infection of the patient.

Numerous applications are concerned with the use of silver salts in antimicrobial coatings which are applied to medical implants or catheters. As compared with metallic silver, silver salts have the disadvantage that in the impregnated coat, alongside the active silver, there are also anions present which under certain circumstances may be toxic, such as nitrate in silver nitrate, for example. A further problem is the rate of release of silver ions from silver salts. Certain silver salts such as silver nitrate are highly soluble in water and are therefore delivered possibly too quickly from the surface coating into the surrounding medium. Other silver salts such as silver chloride are so poorly dissolving that silver ions may be delivered too slowly to the fluid.

Thus U.S. Pat. No. 6,716,895 B1 relates to antimicrobial compositions which as one constituent comprise a hydrophilic polymer which may be selected, among others, from polyether polyurethanes, polyester polyurethanes and polyurethaneureas. The antimicrobial coating is achieved by means of oligodynamic salts, such as through use of silver salts, for example. The composition is used to coat medical devices. A disadvantage of this coating, as well as the aforementioned use of silver salts, is that it is produced starting from a solution of the polymeric constituents, and so in many cases it is not possible to prevent residues of toxic solvent entering the human body following implantation of medical devices which have been provided with this coating.

Further publications, exemplified by WO 2004/017738 A, WO 2001/043788 A and US 2004/0116551 A, describe a concept which involves combining different silver salts to arrive at a silver-containing coating that continuously releases silver ions. The various silver salts are mixed with different polymers, polyurethanes for example, and the combination of silver salts with different water solubilities is tailored in such a way that there is constant release of silver over the entire period in which the coated device is used.

Other processes using silver ions are described by WO 2001/037670 A and US 2003/0147960 A. WO 2001/037670 A describes an antimicrobial formulation which complexes silver ions in zeolites. US 2003/0147960 A describes coatings in which silver ions are bound in a mixture of hydrophilic and hydrophobic polymers.

The processes described that use silver salts have the disadvantages mentioned before, and, moreover, are complicated to implement and therefore expensive in terms of production, and so there continues to be a need for silver-containing coatings that are improved in respect of the production process and the activity.

One interesting possibility for the antimicrobial equipping of plastics is to use nanocrystalline silver particles. The advantage to coating with metallic silver lies in the surface area of the nanocrystalline silver, which is very much greater in relation to its volume; this leads to increased release of silver ions as compared with a metallic silver coating.

Furno et al., Journal of Antimicrobial Chemotherapy 2004, 54, pp. 1019-1024, describe a process which uses supercritical carbon dioxide to impregnate nanocrystalline silver into silicone surfaces. In view of the complex impregnating operation and the obligatory use of supercritical carbon dioxide, this process is expensive and not easy to apply.

In addition there are various known processes for incorporating nanocrystalline silver into plastics. For instance, WO 01/09229 A1, WO 2004/024205 A1, EP 0 711 113 A and Münstedt et al., Advanced Engineering Materials 2000, 2(6), pages 380 to 386 describe the incorporation of nanocrystalline silver into thermoplastic polyurethanes. Pellets of a commercially available thermoplastic polyurethane are soaked in solution with colloidal silver. To increase the antimicrobial activity, WO 2004/024205 A1 and DE 103 51 611 A1 further mention the possibility of using barium sulphate as an additive. Then, from the doped polyurethane pellets, the corresponding products, such as catheters, are produced by extrusion. This procedure, described in the publications, is disadvantageous, furthermore, on account of the fact that the amount of silver which remains on the polyurethane pellets after immersion is not constant and/or cannot be determined beforehand. The effective silver content of the resulting products must therefore be determined afterwards, i.e. after production of the end products. In contrast, a procedure which sets with precision the effective amount of silver to be provided in the resulting end product is not known from these publications.

A similar process is described by EP 0 433 961 A. Here again, a mixture of thermoplastic urethane (Pellethane), silver powder and barium sulphate is mixed and extruded.

A disadvantage of this process is the relatively large amount of silver which is distributed throughout the plastic element. This process is therefore expensive and, as a result of the incorporation of the colloidal silver in the entire plastic matrix, the release of the silver is too slow for sufficient activity in certain cases. The improvement to the release of silver through the addition of barium sulphate represents a further, expensive work-step.

A coating solution comprising a thermoplastic polyurethane with nanocrystalline silver for producing vascular prostheses is described by WO 2006/032497. The structure of the polyurethane is not further specified, but in view of the claiming of thermoplastics, the use of urea-free polyurethanes can be assumed. The antibacterial effect was determined by the growth of adhered Staphylococcus epidermidis cells on the surface of the test element, in comparison with a control. The antibacterial action detected for the silver-containing coatings, however, can be rated as weak, since a retardation of growth by a maximum of only 33.2 h (starting from a defined threshold growth) relative to the control surface was found. For prolonged applications, as an implant or catheter, therefore, this coating formulation is unsuitable.

A further problem is the use of solvents for the preparation of coating solutions. WO 2006/032497 A1 describes an antimicrobial implant having a flexible, porous structure, made from a biocompatible plastic in the form of a nonwoven structure. Components used include solutions of a thermoplastic polyurethane in chloroform. Chloroform is known to be a highly toxic solvent. Where medical products implanted in the human body are coated, there is a risk from residues of this toxic solvent following implantation into the human body.

A further disadvantage of colloidal silver in organic coating solutions is the often low stability of the silver nanoparticles. In organic solutions there may be aggregates of silver particles, and so reproducible silver activity is impossible to formulate. An organic solution to which colloidal silver has been added ought therefore to be processed to completed coatings as soon as possible after its preparation, in order to ensure silver activity consistent from batch to batch. On account of operational practice, however, this procedure is sometimes not possible.

An aqueous polyurethane coating with colloidally distributed silver contained is therefore desirable as an antimicrobial coating.

US 2006/045899 describes antimicrobial formulations with the assistance of aqueous polyurethane systems. The antimicrobial formulation is a mixture of different materials, which makes the manufacture of these products difficult. The nature of the aqueous polyurethane systems is not precisely described, apart from a statement that they are cationically or anionically stabilized dispersions.

CN 1760294 refers likewise to anionic polyurethane dispersions with silver powder having a particle size of 0.2 to 10 μm. According to the prior art, such particle sizes are not sufficient for high antimicrobial activity.

C.-W. Chou et al., Polymer Degradation and Stability 91 (2006), 1017-1024 describes sulphonate-modified dispersions of polyether polyurethanes into which very small amounts (0.00151 % to 0.0113% by weight) of colloidal silver are incorporated. The aim of these studies was to improve the thermal and mechanical properties of the polyurethane employed. No antimicrobial action was investigated, and any such action is unlikely in view of the very small quantities of silver.

Starting out from this prior art, the object of the present invention is to provide medical devices with coatings which exhibit satisfactory antimicrobial activity.

### EMBODIMENTS OF THE INVENTION

An embodiment of the present invention is a medical device comprising a coating obtained from an aqueous dispersion, said aqueous dispersion comprising at least one nonionically stabilized polyurethaneurea and at least one silver-containing constituent.

Another embodiment of the present invention is the above medical device, wherein said at least one nonionically stabilized polyurethaneurea comprises a macropolyol synthesis component selected from the group consisting of polyester polyols, polycarbonate polyols, polyether polyols, and mixtures thereof.

Another embodiment of the present invention is the above medical device, wherein said macropolyol synthesis component is selected from the group consisting of a polyether polyol and a polycarbonate polyol.

Another embodiment of the present invention is the above medical device, wherein the polyurethaneurea of said coating is synthesized from at least the following synthesis components:
a) at least one macropolyol;
b) at least one polyisocyanate;
c) at least one diamine or amino alcohol;
d) at least one monofunctional polyoxyalkylene ether; and
h) antimicrobially active silver.

Another embodiment of the present invention is the above medical device, wherein said at least one silver-containing constituent is a, high-porosity silver powder, silver on support material, or a colloidal silver sol.

Another embodiment of the present invention is the above medical device, wherein said coating comprises nanocrystalline silver particles with an average size in the range of from 1 to 1000 nm are used.

Another embodiment of the present invention is the above medical device, wherein the amount of silver present in said coating, based on the amount of solid nonionically stabilized polyurethaneurea and calculated as Ag and Ag+, is in the range of from 0.1% to 10% by weight.

Yet another embodiment of the present invention is a process for producing a medical device comprising at least one coating, comprising applying an aqueous dispersion comprising at least one nonionically stabilized polyurethaneurea and at least one silver-containing constituent to said medical device.

Another embodiment of the present invention is the above process, comprising applying said aqueous dispersion to said medical device by knifecoating, printing, transfer coating, spraying, spin coating, or dipping.

Yet another embodiment of the present invention is a medical device comprising a coating obtained by the above process.

Another embodiment of the present invention is the above medical device, wherein said medical device is selected from the group consisting of contact lenses; cannulas; catheters; urological catheters; urinary catheters; ureteral catheters; central venous catheters; venous catheters; inlet catheters; outlet catheters; dilation balloons; catheters for angioplasty; catheters for biopsy; catheters for introducing a stent; catheters for introducing an embolism filter; catheters for introducing a vena cava filter; balloon catheters; expandable medical devices; endoscopes; laryngoscopes; tracheal devices; endotracheal tubes; respirators; tracheal aspiration devices; bronchoalveolar lavage catheters; catheters used in coronary angioplasty; guide rods; insertion guides; vascular plugs; pacemaker components; cochlear implants; dental implant tubes for feeding; drainage tubes; guide wires; gloves; stents; implants; extracorporeal blood lines; membranes, dialysis membranes; blood filters; devices for circulatory support; dressing materials for wound management; urine bags; stoma bags; implants which comprise a medically active agent; stents which comprise a medically active agent; balloon surfaces which comprise a medically active agent; contraceptives which comprise a medically active agent; endoscopes; laryngoscopes; and feeding tubes.

### BRIEF DESCRIPTION OF THE DRAWING

FIG. 1 depicts a graph comparing the bacterial adherence of E. coli on different polyurethane surfaces.

### DESCRIPTION OF THE INVENTION

This object is achieved through the provision of medical devices which have at least one coating which is obtained starting from an aqueous dispersion comprising at least one nonionically stabilized polyurethaneurea and at least one silver-containing constituent.

In accordance with the invention it has been found that polyurethaneurea coatings comprising silver exhibit effective release of silver when the polyurethane is nonionically modified and the coating is obtained starting from an aqueous dispersion. Corresponding experiments in accordance with the invention, and also corresponding comparative experiments, which support this finding, are described later on below.

Polyurethaneureas for the purposes of the present invention are polymeric compounds which have

(a) repeat units containing at least two urethane groups, of the following general structure

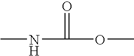

and

(b) at least one repeat unit containing urea groups

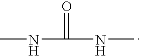

The coating compositions for use in accordance with the invention are based on polyurethaneureas which have substantially no ionic modification. By this is meant, in the context of the present invention, that the polyurethaneureas for use in accordance with the invention have essentially no ionic groups, such as, more particularly, no sulphonate, carboxylate, phosphate and phosphonate groups.

The term “substantially no ionic groups” means, in the context of the present invention, that the resulting coating of the polyurethaneurea contain ionic groups in a fraction of in general not more than 2.50% by weight, more particularly not more than 2.00% by weight, preferably not more than 1.50% by weight, with particular preference not more than 1.00% by weight, especially not more than 0.50% by weight, and even more especially no ionic groups. Hence it is particularly preferred for the polyurethaneurea not to contain any ionic groups.

The polyurethaneureas provided in accordance with the invention for the coating of the medical devices are preferably substantially linear molecules, but may also be branched, though this is less preferred. By substantially linear molecules are meant systems with a slight degree of incipient crosslinking, comprising a macropolyol component as a synthesis component, preferably selected from the group consisting of a polyether polyol, a polycarbonate polyol and a polyester polyol, which have an average functionality of preferably 1.7 to 2.3, more particularly 1.8 to 2.2, more preferably 1.9 to 2.1.

If mixtures of macropolyols and, if appropriate, polyols have been used in the polyurethaneurea as elucidated in more detail below, then the average functionality refers to an average value arising from the totality of the macropolyols and/or polyols.

The number-average molecular weight of the polyurethaneureas used with preference in accordance with the invention is preferably 1000 to 200 000, more preferably from 5000 to 100 000. The number-average molecular weight here is measured against polystyrene as standard in dimethylacetamide at 30° C.

### Polyurethaneureas

The polyurethaneurea-based coating systems for use in accordance with the invention are described in more detail below.

The polyurethaneureas used in accordance with the invention in the coatings of medical devices are formed by reaction of at least one macropolyol component, at least one polyisocyanate component, at least one polyoxyalkylene ether, at least one diamine and/or amino alcohol and, if desired, a polyol component. Further synthesis components may be present in the polyurethaneurea of the invention.

### (a) Macropolyol Component

The composition of the polyurethaneurea coating provided in accordance with the invention has units which derive from at least one macropolyol component as a synthesis component.

This macropolyol component is generally selected from the group consisting of a polyether polyol, a polycarbonate polyol, a polyester polyol and any desired mixtures of these.

In one preferred embodiment of the present invention the synthesis component of a polyol is formed of a polyether polyol or a polycarbonate polyol and also of mixtures of polyether polyol and a polycarbonate polyol.

In a further embodiment of the present invention the synthesis component of a macropolyol is formed of a polyether polyol, more particularly a polyether diol. Polyether polyols and, in particular, polyether diols are particularly preferred in respect of the release of silver. Corresponding experiments according to the invention that support these findings are shown later on below.

In the text below, the individual macropolyol synthesis components are described in more detail, the present invention embracing polyurethaneureas which comprise only one synthesis component, selected from, in general, polyether polyols, polyester polyols, and polycarbonate polyols, and also embracing mixtures of these synthesis components. Furthermore, the polyurethaneureas provided in accordance with the invention may also comprise one or more different representatives of these classes of synthesis components.

The above-defined functionality of the polyurethaneureas provided in accordance with the invention is understood, where two or more different macropolyols and polyols or polyamines (which are described further on below under c) and e)) are present in the polyurethaneurea, to be the average functionality.

### Polyether Polyol

The hydroxyl-containing polyethers in question are those which are prepared by polymerizing cyclic ethers such as ethylene oxide, propylene oxide, butylene oxide, tetrahydrofuran, styrene oxide or epichlorohydrin with themselves, in the presence of BF3 or of basic catalysts, for example, or by addition reaction of these ring compounds, where appropriate in a mixture or in succession, with starter components containing reactive hydrogen atoms, such as alcohols and amines or amino alcohols, e.g. water, ethylene glycol, propylene 1,2-glycol or propylene 1,3-glycol.

Preferred hydroxyl-containing polyethers are those based on ethylene oxide, propylene oxide or tetrahydrofuran or on mixtures of these cyclic ethers. Especially preferred hydroxyl-containing polyethers are those based on polymerized tetrahydrofuran. It is also possible to add other hydroxyl-containing polyethers such as those based on ethylene oxide or propylene oxide, but in that case the polyethers based on tetrahydrofuran are present at, preferably, 50% by weight at least.

### Polycarbonate Polyol

Suitable in principle for the introduction of units based on a hydroxyl-containing polycarbonate are polyhydroxy compounds having an average hydroxyl functionality of 1.7 to 2.3, preferably of 1.8 to 2.2, more preferably of 1.9 to 2.1.

Suitable hydroxyl-containing polycarbonates are polycarbonates of a molecular weight, as determined through the OH number, of preferably 400 to 6000 g/mol, more preferably 500 to 5000 g/mol, more particularly of 600 to 3000 g/mol, which are obtainable, for example, through reaction of carbonic acid derivatives, such as diphenyl carbonate, dimethyl carbonate or phosgene, with polyols, preferably diols. Examples of suitable such diols include ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, di-, tri- or tetraethylene glycol, dipropylene glycol, polypropylene glycols, dibutylene glycol, polybutylene glycols, bisphenol A, tetrabromobisphenol A, and also lactone-modified diols.

The diol component preferably contains 40% to 100% by weight of hexanediol, preferably 1,6-hexanediol and/or hexanediol derivatives, preferably those which as well as terminal OH groups contain ether or ester groups, examples being products obtained by reaction of 1 mol of hexanediol with at least 1 mol, preferably 1 to 2 mol, of caprolactone or through etherification of hexanediol with itself to give the di- or trihexylene glycol. Polyether-polycarbonate diols as well can be used. The hydroxyl polycarbonates ought to be substantially linear. If desired, however, they may be slightly branched as a result of the incorporation of polyfunctional components, more particularly low molecular weight polyols. Examples of those suitable for this purpose include glycerol, timethylolpropane, hexane-1,2,6-triol, butane-1,2,4-triol, trimethylolpropane, pentaerythritol, quinitol, mannitol, sorbitol, methylglycoside or 1,3,4,6-dianhydrohexitols. Preferred polycarbonates are those based on hexane-1,6-diol, and also on co-diols with a modifying action such as butane-1,4-diol, for example, or else on ε-caprolactone. Further preferred polycarbonate diols are those based on mixtures of hexane-1,6-diol and butane-1,4-diol.

The polycarbonate is preferably substantially linear in construction and has only a slight three-dimensional crosslinking, with the consequence that polyurethanes are formed which have the specification identified above.

### Polyester Polyol

The hydroxyl-containing polyesters that are suitable are, for example, reaction products of polyhydric, preferably dihydric, alcohols with polybasic, preferably dibasic, polycarboxylic acids. In place of the free carboxylic acids it is also possible to use the corresponding polycarboxylic anhydrides or corresponding polycarboxylic esters of lower alcohols, or mixtures thereof, to prepare the polyesters.

The polycarboxylic acids may be aliphatic, cycloaliphatic, aromatic and/or heterocyclic in nature and may if appropriate be substituted, by halogen atoms, for example, and/or unsaturated. Aliphatic and cycloaliphatic dicarboxylic acids are preferred. Examples thereof include the following:

succinic acid, adipic acid, azelaic acid, sebacic acid, phthalic acid, tetrachlorophthalic acid, isophthalic acid, terephthalic acid, tetrahydrophthalic acid, hexahydrophthalic acid, cyclohexanedicarboxylic acid, itaconic acid, sebacic acid, glutaric acid, suberic acid, 2-methylsuccinic acid, 3,3-diethylglutaric acid, 2,2-dimethylsuccinic acid, maleic acid, malonic acid, fumaric acid or dimethyl terephthalate. Anhydrides of these acids can likewise be used, where they exist. Examples thereof are maleic anhydride, phthalic anhydride, tetrahydrophthalic anhydride, glutaric anhydride, hexahydrophthalic anhydride and tetrachlorophthalic anhydride.

As a polycarboxylic acid for use in small amounts, if appropriate, mention may be made here of trimellitic acid.

The polyhydric alcohols employed are preferably diols. Examples of such diols are, for example, ethylene glycol, propylene 1,2-glycol, propylene 1,3-glycol, butane-1,4-diol, butane-2,3-diol, diethylene glycol, triethylene glycol, hexane-1,6-diol, octane-1,8-diol, neopentyl glycol, 2-methyl-1,3-propanediol or neopentyl glycol hydroxypivalate. Polyester diols formed from lactones, ε-caprolactone for example, can also be employed. Examples of polyols which can be used as well if appropriate are trimethylolpropane, glycerol, erythritol, pentaerythritol, trimethylolbenzene or trishydroxyethyl isocyanurate.

### (b) Polyisocyanate

The composition of the polyurethaneurea coating provided in accordance with the invention comprises units which originate from at least one polyisocyanate as a synthesis component.

As polyisocyanates (b) it is possible to use all of the aromatic, araliphatic, aliphatic and cycloaliphatic isocyanates that are known to the skilled person and have an average NCO functionality ≧1, preferably ≧2, individually or in any desired mixtures with one another, irrespective of whether they have been prepared by phosgene or phosgene-free processes. They may also contain iminooxadiazinedione, isocyanurate, uretdione, urethane, allophanate, biuret, urea, oxadiazinetrione, oxazolidinone, acylurea and/or carbodiimide structures. The polyisocyanates may be used individually or in any desired mixtures with one another.

Preference is given to using isocyanates from the series of the aliphatic or cycloaliphatic representatives, which have a carbon backbone (without the NCO groups present) of 3 to 30, preferably 4 to 20, carbon atoms.

Particularly preferred compounds of component (b) conform to the type specified above having aliphatically and/or cycloaliphatically attached NCO groups, such as, for example, bis(isocyanatoalkyl) ethers, bis- and tris(isocyanatoalkyl)benzenes, -toluenes, and -xylenes, propane diisocyanates, butane diisocyanates, pentane diisocyanates, hexane diisocyanates (e.g. hexamethylene diisocyanate, HDI), heptane diisocyanates, octane diisocyanates, nonane diisocyanates (e.g. trimethyl-HDI (TMDI), generally as a mixture of the 2,4,4 and 2,2,4 isomers), nonane triisocyanates (e.g. 4-isocyanatomethyl-1,8-octane diisocyanate), decane diisocyanates, decane triisocyanates, undecane diisocyanates, undecane triisocyanates, dodecane diisocyanates, dodecane triisocyanates, 1,3- and 1,4-bis(iso-cyanatomethyl)cyclohexanes (H₆XDI), 3-isocyanatomethyl-3,5,5-trimethylcyclohexyl isocyanate (isophorone diisocyanate, IPDI), bis(4-isocyanatocyclohexyl)methane (H₁₂MDI) or bis(isocyanatomethyl)norbornane (NBDI).

Very particularly preferred compounds of component (b) are hexamethylene diisocyanate (HDI), trimethyl-HDI (TMDI), 2-methylpentane 1,5-diisocyanate (MPDI), isophorone diisocyanate (IPDI), 1,3- and 1,4-bis(isocyanatomethyl)cyclohexane (H₆XDI), bis(isocyanatomethyl)norbornane (NBDI), 3(4)-isocyanatomethyl-1-methylcyclohexyl isocyanate (IMCI) and/or 4,4′-bis(isocyanatocyclohexyl)methane (H₁₂MDI) or mixtures of these isocyanates. Further examples are derivatives of the above diisocyanates with a uretdione, isocyanurate, urethane, allophanate, biuret, iminooxadiazinedione and/or oxadiazinetrione structure and with more than two NCO groups.

The amount of constituent (b) in the coating of the invention is preferably 1.0 to 3.5 mol, more preferably 1.0 to 3.3 mol, more particularly 1.0 to 3.0 mol, based in each case on the constituent (a) of the coating for use in accordance with the invention.

### (c) Diamine or Amino Alcohol

The composition of the polyurethaneurea coating provided in accordance with the invention comprises units which originate from at least one diamine or amino alcohol as a synthesis component and serve as what are called chain extenders (c).

Such chain extenders are, for example, diamines or polyamines and also hydrazides, e.g. hydrazine, 1,2-ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, 1,3- and 1,4-xylylenediamine, α,α,α′,α′-tetramethyl-1,3- and -1,4-xylylenediamine and 4,4′-diaminodicyclohexylmethane, dimethylethylenediamine, hydrazine, adipic dihydrazide, 1,4-bis-(aminomethyl)cyclohexane, 4,4′-diamino-3,3′-dimethyldicyclohexylmethane and other (C1-C4) di- and tetraalkyldicyclohexylmethanes, e.g. 4,4′-diamino-3,5-diethyl-3′,5′-diisopropyldicyclohexylmethane.

Suitable diamines or amino alcohols are generally low molecular weight diamines or amino alcohols which contain active hydrogen with differing reactivity towards NCO groups, such as compounds which as well as a primary amino group also contain secondary amino groups or which as well as an amino group (primary or secondary) also contain OH groups. Examples of such compounds are primary and secondary amines, such as 3-amino-1-methylaminopropane, 3-amino-1-ethylaminopropane, 3-amino-1-cyclohexylaminopropane, 3-amino-1-methylaminobutane, and also amino alcohols, such as N-aminoethylethanolamine, ethanolamine, 3-aminopropanol, neopentanolamine and, with particular preference, diethanolamine.

The constituent (c) of the coating composition for use in accordance with the invention can be used, in the context of the preparation of the coating composition, as a chain extender.

The amount of constituent (c) in the coating composition for use in accordance with the invention is preferably 0.1 to 1.5 mol, more preferably 0.2 to 1.3 mol, more particularly 0.3 to 1.2 mol, based in each case on the constituent (a) of the coating composition for use in accordance with the invention.

### (d) Polyoxyalkylene Ethers

The polyurethaneurea used in the present invention has units which originate from a polyoxyalkylene ether as a synthesis component.

The polyoxyalkylene ether is preferably a copolymer of polyethylene oxide and polypropylene oxide. These copolymer units are present in the form of end groups in the polyurethaneurea, and have the effect of making the coating composition of the invention hydrophilic.

Suitable nonionically hydrophilicizing compounds meeting the definition of component (d) are, for example, polyoxyalkylene ethers which contain at least one hydroxyl or amino group. These polymers contain in general a fraction of 30% to 100% by weight of units derived from ethylene oxide.

Nonionically hydrophilicizing compounds (d) are, for example, monofunctional polyalkylene oxide polyether alcohols containing on average 5 to 70, preferably 7 to 55, ethylene oxide units per molecule, of the kind available in conventional manner through alkoxylation of suitable starter molecules (e.g. in Ullmanns Enzyklopädie der technischen Chemie, 4th Edition, Volume 19, Verlag Chemie, Weinheim pp. 31-38).

Examples of suitable starter molecules are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as diethylene glycol monobutyl ether, for example, unsaturated alcohols such as allyl alcohol, 1,1-dimethylallyl alcohol or oleyl alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexylamine, N-methyl- and N-ethylcyclohexylamine or dicyclohexylamine, and also heterocyclic secondary amines such as morpholine, pyrrolidine, piperidine or 1H-pyrazole. Preferred starter molecules are saturated monoalcohols. Particular preference is given to using diethylene glycol monobutyl ether as a starter molecule.

Alkylene oxides suitable for the alkoxylation reaction are, in particular, ethylene oxide and propylene oxide, which can be used in any order or else in a mixture in the alkoxylation reaction.

The polyalkylene oxide polyether alcohols are either pure polyethylene oxide polyethers or mixed polyalkylene oxide polyethers whose alkylene oxide units are composed to an extent of at least 30 mol %, preferably at least 40 mol %, of ethylene oxide units. Preferred nonionic compounds are monofunctional mixed polyalkylene oxide polyethers which contain at least 40 mol % of ethylene oxide units and not more than 60 mol % of propylene oxide units.

When the alkylene oxides ethylene oxide and propylene oxide are used, they can be used in any order or else in a mixture in the alkoxylation reaction.

The average molar weight of the polyoxyalkylene ether is preferably 500 g/mol to 5000 g/mol, more preferably 1000 g/mol to 4000 g/mol, more particularly 1000 to 3000 g/mol.

The amount of constituent (d) in the coating composition for use in accordance with the invention is preferably 0.01 to 0.5 mol, more preferably 0.02 to 0.4 mol, more particularly 0.04 to 0.3 mol, based in each case on the constituent (a) of the coating composition for use in accordance with the invention.

### (e) Polyols

In a further embodiment the composition of the polyurethaneurea coating provided in accordance with the invention further comprises units which originate from at least one polyol as a synthesis component. These polyol synthesis components, in comparison to the macropolyol, are relatively short-chain synthesis components, which through additional hard segments can give rise to stiffening.

The low molecular weight polyols (e) used to synthesize the polyurethaneureas have the effect, generally, of stiffening and/or branching the polymer chain. The molecular weight is preferably 62 to 500 g/mol, more preferably 62 to 400 g/mol, more particularly 62 to 200 g/mol.

Suitable polyols may contain aliphatic, alicyclic or aromatic groups. Mention may be made here, for example, of the low molecular weight polyols having up to about 20 carbon atoms per molecule, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), and also trinethylolpropane, glycerol or pentaerythritol, and mixtures of these and, if desired, other low molecular weight polyols as well. Use may also be made of ester diols such as, for example, α-hydroxybutyl-ε-hydroxycaproic acid ester, ω-hydroxyhexyl-γ-hydroxybutyric acid ester, adipic acid β-hydroxyethyl ester or terephthalic acid bis(β-hydroxyethyl) ester.

The amount of constituent (e) in the coating composition for use in accordance with the invention is preferably 0.05 to 1.0 mol, more preferably 0.05 to 0.5 mol, more particularly 0.1 to 0.5 mol, based in each case on the constituent (a) of the coating composition for use in accordance with the invention.

(f) Further Amine- and/or Hydroxy-Containing Units (Synthesis Component)

The reaction of the isocyanate-containing component (b) with the hydroxy- or amine-functional compounds (a), (c), (d) and, if used, (e) takes place typically with a slight NCO excess observed over the reactive hydroxy or amine compounds. In this case, at the end point of the reaction, through attainment of a target viscosity, there still always remain residues of active isocyanate. These residues must be blocked so that there is no reaction with large polymer chains. Such a reaction leads to the three-dimensional crosslinking and gelling of the batch. The processing of such a coating solution is no longer possible, or is possible only with restrictions. The batches typically contain large amounts of water. Over the course of a number of hours, on standing or on stirring of the batch at room temperature, the water causes the isocyanate groups that still remain to be consumed by reaction.

If, however, the desire is to block the remaining, residual isocyanate content rapidly, the polyurethaneurea coatings provided in accordance with the invention may also comprise monomers (f), which are located in each case at the chain ends and cap them.

These units derive on the one hand from monofunctional compounds that are reactive with NCO groups, such as monoamines, more particularly mono-secondary amines, or monoalcohols. Mention may be made here, for example, of ethanol, n-butanol, ethylene glycol monobutyl ether, 2-ethylhexanol, 1-octanol, 1-dodecanol, 1-hexadecanol, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof.

Since the units (f) are used essentially in the coatings of the invention to destroy the NCO excess, the amount required is dependent essentially on the amount of the NCO excess, and cannot be specified generally.

Preferably, these units are not used during the synthesis. In that case, unreacted isocyanate is hydrolysed, preferably, by the dispersing water.

### (g) Further Constituents

Although the antibacterial (antimicrobial) polyurethaneurea coating already provides the medical devices of the invention with sufficient functionalization, it may be of advantage in a specific case to integrate further functionalizations into the coating. These further possible functionalizations are now described in more detail below.

Furthermore, the polyurethaneurea coatings provided in accordance with the invention may comprise further constituents typical for the intended purpose, such as additives and fillers. An example of such are active pharmacological substances, medicaments and additives which promote the release of active pharmacological substances (drug-eluting additives).

Active pharmacological substances and medicaments which may be used in the coatings of the invention on the medical devices are in general, for example, thromboresistant agents, antibiotic agents, antitumour agents, growth hormones, antiviral agents, antiangiogenic agents, angiogenic agents, antimitotic agents, anti-inflammatory agents, cell cycle regulators, genetic agents, hormones, and also their homologues, derivatives, fragments, pharmaceutical salts, and combinations thereof.

Specific examples of such medicaments and active pharmacological substances hence include thromboresistant (non-thrombogenic) agents and other agents for suppressing acute thrombosis, stenosis or late restenosis of the arteries, examples being heparin, streptokinase, urokinase, tissue plasminogen activator, anti-thromboxan-B₂ agent; anti-B-thromboglobulin, prostaglandin-E, aspirin, dipyridimol, anti-thromboxan-A₂ agent, murine monoclonal antibody 7E3, triazolopyrimidine, ciprostene, hirudin, ticlopidine, nicorandil, etc. A growth factor can likewise be utilized as a medicament in order to suppress subintimal fibromuscular hyperplasia at the arterial stenosis site, or any other cell growth inhibitor can be utilized at the stenosis site.

The medicament or active pharmacological substance may also be composed of a vasodilatator, in order to counteract vasospasm—for example, an antispasm agent such as papaverine. The medicament may be a vasoactive agent per se, such as calcium antagonists, or α- and β-adrenergic agonists or antagonists. In addition the therapeutic agent may be a biological adhesive such as cyanoacrylate in medical grade, or fibrin, which is used, for example, for bonding a tissue valve to the wall of a coronary artery.

The therapeutic agent may further be an antineoplastic agent such as 5-fluorouracil, preferably with a controlling releasing vehicle for the agent (for example, for the use of an ongoing controlled releasing antineoplastic agent at a tumour site).

The therapeutic agent may be an antibiotic, preferably in combination with a controlling releasing vehicle for ongoing release from the coating of a medical device at a localized focus of infection within the body. Similarly, the therapeutic agent may comprise steroids for the purpose of suppressing inflammation in localized tissue, or for other reasons.

Specific examples of suitable medicaments include:

(a) heparin, heparin sulphate, hirudin, hyaluronic acid, chondroitin sulphate, dermatan sulphate, keratan sulphate, lytic agents, including urokinase and streptokinase, their homologues, analogues, fragments, derivatives and pharmaceutical salts thereof;

(b) antibiotic agents such as penicillins, cephalosporins, vacomycins, aminoglycosides, quinolones, polymyxins, erythromycins; tetracyclines, chloramphenicols, clindamycins, lincomycins, sulphonamides, their homologues, analogues, derivatives, pharmaceutical salts and mixtures thereof;

(c) paclitaxel, docetaxel, immunosuppressants such as sirolimus or everolimus, alkylating agents, including mechlorethamine, chlorambucil, cyclophosphamide, melphalane and ifosfamide; antimetabolites, including methotrexate, 6-mercaptopurine, 5-fluorouracil and cytarabine; plant alkoids, including vinblastin; vincristin and etoposide; antibiotics, including doxorubicin, daunomycin, bleomycin and mitomycin; nitrosurea, including carmustine and lomustine; inorganic ions, including cisplatin; biological reaction modifiers, including interferon; angiostatins and endostatins; enzymes, including asparaginase; and hormones, including tamoxifen and flutamide, their homologues, analogues, fragments, derivatives, pharmaceutical salts and mixtures thereof; and

(d) antiviral agents such as amantadine, rimantadine, rabavirin, idoxuridine, vidarabin, trifluridine, acyclovir, ganciclovir, zidovudine, phosphonoformates, interferons, their homologues, analogues, fragments, derivatives, pharmaceutical salts and mixtures thereof; and

e) antiflammatory agents such as, for example, ibuprofen, dexamethasone or methylprednisolone.

Further typical additives and auxiliaries such as thickeners, hand assistants, pigments, dyes, matting agents, UV stabilizers, phenolic antioxidants, light stabilizers, hydrophobicizing agents and/or flow control assistants may likewise be used in the coating provided in accordance with the invention.

### (h) Antimicrobial Silver

The polyurethaneurea dispersion used in accordance with the invention comprises, besides the polyurethaneurea, at least one silver-containing constituent.

By “a silver-containing constituent” is meant, for the purposes of the present invention, any component capable of releasing silver in elemental or ionic form and hence leading to an antimicrobial (biocidal/antibacterial) effect.

The biocidal effect of silver derives from the interaction of silver ions with bacteria. In order to be able to generate the maximum number of silver ions from elemental silver, a large surface area of the silver is advantageous. Consequently, for antimicrobial applications, use is made primarily of high-porosity silver powders, silver on support materials or colloidal silver sols.

Available commercially at present, for example, are Ag-Ion (silver in a zeolite, Agion, Wakefield, Mass., USA), Ionpure® (Ag+in glass, Ciba Spezialitätenchemie GmbH, Lampertheim, Germany), Alphasan® (AgZr phosphate, Milliken Chemical, Gent, Belgium), Irgaguard® (Ag in zeolite/glass), Hygate® (silver powder, Bio-Gate, Nuremberg, Germany), NanoSilver® BG (silver in suspension) and Nanocid® (silver on TiO2, Pars Nano Nasb Co., Tehran, Iran).

The silver powders are preferably obtained from a gas phase, a silver melt being vaporized in helium. The resultant nanoparticles agglomerate immediately and are obtained in the form of high-porosity, readily filterable powders. The disadvantage of these powders, however, is that the agglomerates can no longer be dispersed into individual particles.

Colloidal silver dispersions are obtained by reducing silver salts in organic or aqueous medium. Their preparation is more complex than that of the silver powders, but offers the advantage that unagglomerated nanoparticles are obtained. As a result of the incorporation of unagglomerated nanoparticles into coating materials it is possible to produce transparent films.

For the silver-containing polyurethaneurea coatings of the invention it is possible to use any desired silver powders or colloidal silver dispersions. A multiplicity of such silver materials is available commercially.

The silver sols used preferably to formulate the silver-containing aqueous polyurethane dispersions of the invention are prepared from A_{g2}O by reduction with a reducing agent such as aqueous formaldehyde solution following prior addition of a dispersing assistant. For this purpose the A_{g2}O sols are prepared, for example, batchwise, by rapid mixing of silver nitrate solution with NaOH, by means of rapid stirring, or in a continuous operation, by using a micromixer conforming to DE 10 2006 017 696. Thereafter the A_{g2}O nanoparticles are reduced with formaldehyde in excess, in a batch process, and, finally, are purified by centrifuging or membrane filtration, preferably by membrane filtration. This mode of production is particularly advantageous on account of the fact that it allows the amount of organic auxiliaries bound on the surface of the nanoparticles to be minimized. The product is a silver sol dispersion in water with an average particle size of approximately 10 to 150 nm, more preferably 20 to 100 nm.

For the production of antimicrobially equipped coatings it is possible to use nanocrystalline silver particles with an average size of 1 to 1000 nm, preferably 5 to 500 nm, very preferably of 10 to 250 nm. The silver nanoparticles can be dispersed in organic solvents or water, preferably in water-miscible organic solvents or water, very preferably in water. The raw coating materials are prepared by adding the silver dispersion to the polyurethane solution and then carrying out homogenization by stirring or shaking.

The amount of nanocrystalline silver, based on the amount of solid polymer and calculated as Ag and Ag+, can be varied. Typical concentrations range from 0.1% to 10%, preferably from 0.3% to 5%, more preferably from 0.5% to 3%, by weight.

In comparison to many alternative processes, the advantage of the silver-containing polyurethanes of the invention lies in the great ease of combination of the aqueous polyurethane dispersions and the aqueous colloidal silver dispersions. Different silver concentrations can be set easily and precisely for different applications in accordance with requirements. Many processes of the prior art are substantially more complicated and are also not so precise in the metering of the amount of silver as the process of the invention.

In one particularly preferred embodiment the antimicrobial silver is in the form of high-porosity silver powders, silver on support materials, or in the form of colloidal silver sols, with 0.1% to 10% by weight of silver being present, based on the solid polyurethane polymer.

In a further particularly preferred embodiment the antimicrobial silver is in the form of colloidal silver sols in aqueous medium or in water-miscible organic solvents, with a particle size of 1 to 1000 nm, the amount added being 0.3% to 5% by weight, based on the solid polyurethane polymer.

In a further particularly preferred embodiment, the antimicrobial silver is in the form of colloidal silver sols in aqueous medium, with an average particle size of 1 to 500 nm, the amount added being 0.5% to 3% by weight, based on the solid polyurethane polymer.

### Coating Composition

In one preferred embodiment the coating composition provided in accordance with the invention comprises a polyurethaneurea which is synthesized at least from
a) at least one macropolyol;
b) at least one polyisocyanate;
c) at least one diamine or amino alcohol; and
d) at least one monofunctional polyoxyalkylene ether; and also
h) at least one antimicrobial, silver-containing constituent.

In a further embodiment of the present invention the coating composition provided in accordance with the invention comprises a polyurethaneurea which is synthesized at least from
a) at least one macropolyol;
b) at least one polyisocyanate;
c) at least one diamine or amino alcohol;
d) at least one monofunctional polyoxyalkylene ether; and
e) at least one further polyol; and also
h) at least one antimicrobial silver-containing constituent.

In a further embodiment of the present invention the coating composition provided in accordance with the invention comprises a polyurethaneurea which is synthesized at least from
a) at least one macropolyol;
b) at least one polyisocyanate;
c) at least one diamine or amino alcohol;
d) at least one monofunctional polyoxyalkylene ether;
e) at least one further polyol; and
f) at least one amine- or hydroxyl-containing monomer which is located at the polymer chain ends; and also
h) at least one antimicrobial silver-containing constituent.

Particularly preferred in accordance with the invention for coating the medical devices are polyurethaneurea dispersions which are synthesized from

a) at least one macropolyol having an average molar weight between 400 g/mol and 6000 g/mol and a hydroxyl functionality of 1.7 to 2.3;

b) at least one aliphatic, cycloaliphatic or aromatic polyisocyanate, or mixtures of such polyisocyanates, in an amount of 1.0 to 3.5 mol per mole of the macropolyol;

c) at least one aliphatic or cycloaliphatic diamine or at least one amino alcohol as so-called chain extender, or mixtures of such compounds, in an amount of 0.1 to 1.5 mol per mole of the macropolyol;

d) at least one monofunctional polyoxyalkylene ether, or a mixture of such polyethers, with an average molar weight between 500 g/mol and 5000 g/mol, in an amount of 0.01 to 0.5 mol per mole of the macropolyol;

e) if desired, one or more short-chain aliphatic polyols having a molar weight between 62 g/mol and 500 g/mol, in an amount of 0.05 to 1 mol per mole of the macropolyol; and

f) if desired, amine- or OH-containing units which are located on, and cap, the polymer chain ends; and also

h) at least one antimicrobial, silver-containing constituent.

Further preferred in accordance with the invention for coating medical devices are polyurethaneurea dispersions which are synthesized from
a) at least one macropolyol having an average molar weight between 500 g/mol and 5000 g/mol and a hydroxyl functionality of 1.8 to 2.2;
b) at least one aliphatic, cycloaliphatic or aromatic polyisocyanate, or mixtures of such polyisocyanates, in an amount of 1.0 to 3.3 mol per mole of the macropolyol;
c) at least one aliphatic or cycloaliphatic diamine or at least one amino alcohol as so-called chain extender, or mixtures of such compounds, in an amount of 0.2 to 1.3 mol per mole of the macropolyol;
d) at least one monofunctional polyoxyalkylene ether, or a mixture of such polyethers, with an average molar weight between 1000 g/mol and 4000 g/mol, in an amount of 0.02 to 0.4 mol per mole of the macropolyol;
e) if desired, one or more short-chain aliphatic polyols having a molar weight between 62 g/mol and 400 g/mol, in an amount of 0.05 to 0.5 mol per mole of the macropolyol; and
f) if desired, amine- or OH-containing units which are located on, and cap, the polymer chain ends; and also
h) at least one antimicrobial, silver-containing constituent.

Even further preferred in accordance with the invention for coating medical devices are polyurethaneurea dispersions which are synthesized from
a) at least one macropolyol having an average molar weight between 600 g/mol and 3000 g/mol and a hydroxyl functionality of 1.9 to 2.1;
b) at least one aliphatic, cycloaliphatic or aromatic polyisocyanate, or mixtures of such polyisocyanates, in an amount of 1.0 to 3.0 mol per mole of the macropolyol;
c) at least one aliphatic or cycloaliphatic diamine or at least one amino alcohol as so-called chain extender, or mixtures of such compounds, in an amount of 0.3 to 1.2 mol per mole of the macropolyol;
d) at least one monofunctional polyoxyalkylene ether, or a mixture of such polyethers, with an average molar weight between 1000 g/mol and 3000 g/mol, in an amount of 0.04 to 0.3 mol per mole of the macropolyol, more particular preference being given to a mixture of polyethylene oxide and polypropylene oxide; and
e) if desired, one or more short-chain aliphatic polyols having a molar weight between 62 g/mol and 400 g/mol, in an amount of 0.1 to 0.5 mol per mole of the macropolyol; and also
h) at least one antimicrobial, silver-containing constituent.

### Medical Device

The term “medical device” is to be understood broadly in the context of the present invention. Suitable, non-limiting examples of medical devices (including instruments) are contact lenses; cannulas; catheters, for example urological catheters such as urinary catheters or ureteral catheters; central venous catheters; venous catheters or inlet or outlet catheters; dilation balloons; catheters for angioplasty and biopsy; catheters used for introducing a stent, an embolism filter or a vena cava filter; balloon catheters or other expandable medical devices; endoscopes; laryngoscopes; tracheal devices such as endotracheal tubes, respirators and other tracheal aspiration devices; bronchoalveolar lavage catheters; catheters used in coronary angioplasty; guide wires, insertion guides and the like; vascular plugs; pacemaker components; cochlear implants; dental implant tubes for feeding, drainage tubes; and guide wires.

The coating solutions of the invention may be used, furthermore, for producing protective coatings, for example for gloves, stents and other implants; external (extracorporeal) blood lines (blood-carrying pipes); membranes; for example for dialysis; blood filters; devices for circulatory support; dressing material for wound management; urine bags and stoma bags. Also included are implants which comprise a medically active agent, such as medically active agents for stents or for balloon surfaces or for contraceptives.

Typically the medical device is formed from catheters, endoscopes, laryngoscopes, endotracheal tubes, feeding tubes, guide rods, stents, and other implants.

There are many materials suitable as a substrate of the surface to be coated, such as metals, textiles, ceramics or plastics, the use of metals and plastics being preferred for the production of medical devices.

Examples of metals may include the following: medical stainless steel or nickel-titanium alloys.

In the case of catheters, these are preferably made from plastics such as polyamide, block copolymers of styrene and unsaturated compounds such as ethylene, butylene and isoprene, polyethylene or copolymers of polyethylene and polypropylene, silicone, polyvinyl chloride (PVC) and/or polyurethanes. For improved adhesion of the coating compositions that are essential to the invention, the surface of the medical article may have been subjected beforehand to a surface treatment such as coating with an adhesion promoter.

Production of the Coating Dispersion used in Accordance with the Invention

The stated synthesis components (a), (b), (d) and, if desired, (e) are reacted so as to prepare, first of all, an isocyanate-functional prepolymer which is free of urea groups, the amount-of-substance ratio of isocyanate groups to isocyanate-reactive groups being 0.8 to 3.5, preferably 0.9 to 3.0, more preferably 1.0 to 2.5, and subsequently the remaining isocyanate groups are given an amino-functional chain extension or chain termination, before, during or after dispersion in water, the ratio of equivalents of isocyanate-reactive groups of the compounds used for chain extension to free isocyanate groups of the prepolymer being between 40% to 150%, preferably between 50% to 120%, more preferably between 60% to 120%.

The polyurethaneurea dispersions of the invention which serve as starting material for the production of the coatings of the invention are prepared preferably by the process known as the acetone process.

For the preparation of the polyurethaneurea dispersions by this acetone process, typically, some or all of the synthesis components (a), (d) and, if desired, (e), which must not contain any primary or secondary amino groups, and the polyisocyanate component (b) for preparing an isocyanate-functional polyurethane prepolymer, are introduced and, where appropriate, are diluted with a water-miscible solvent which is nevertheless inert towards isocyanate groups, and this initial charge is heated to temperatures in the range from 50 to 120° C. To accelerate the isocyanate addition reaction it is possible to use the catalysts known in polyurethane chemistry, an example being dibutyltin dilaurate. Preference is given to synthesis without catalyst.

Suitable solvents are the typical aliphatic, keto-functional solvents such as, for example, acetone, butanone, which can be added not only at the beginning of the preparation but also, if desired, in portions later on as well. Acetone and butanone are preferred. Other solvents such as xylene, toluene, cyclohexane, butyl acetate, methoxypropyl acetate, and solvents with ether units or ester units, for example, may likewise be used and may be removed in whole or in part by distillation or may remain entirely in the dispersion.

Subsequently any constituents from (a), (b), (d) and, if used, (e) not added at the beginning of the reaction are metered in.

In the preparation of the polyurethane prepolymer the amount-of-substance ratio of isocyanate groups to isocyanate-reactive groups is 0.8 to 3.5, preferably 0.9 to 3.0, more preferably 1.0 to 2.5.

The reaction of components (a), (b), (d) and, if used, (e) to give the prepolymer takes place partially or completely, but preferably completely. In this way, polyurethane prepolymers which contain free isocyanate groups are obtained, in bulk or in solution.

Subsequently, in a further process step, if it has not yet taken place or has taken place only partly, the resulting prepolymer is dissolved by means of aliphatic ketones such as acetone or butanone.

Subsequently, possible NH₂- and/or NH-functional components are reacted with the remaining isocyanate groups. This chain extension/termination can be carried out alternatively in solvent prior to dispersing, during dispersing, or in water after dispersing has taken place. Preference is given to carrying out the chain extension prior to dispersing in water.

Where compounds conforming to the definition of (c) with NH₂ or NH groups are used for chain extension, the chain extension of the prepolymers takes place preferably prior to dispersing.

The degree of chain extension, in other words the ratio of equivalents of NCO-reactive groups of the compounds used for chain extension to free NCO groups of the prepolymer, is between 40% to 150%, preferably between 50% to 120%, more preferably between 60% to 120%.

The aminic components (c) may if desired be used in water-diluted or solvent-diluted form in the process of the invention, individually or in mixtures, in which case any sequence of addition is possible in principle. If water or organic solvents are used as diluents, the diluent content is preferably 70% to 95% by weight.

The preparation of the polyurethane dispersion from the prepolymers takes place following the chain extension. For this purpose, either the dissolved and chain-extended polyurethane polymer is introduced into the dispersing water, where appropriate with strong shearing, such as vigorous stirring, for example, or, conversely, the dispersing water is stirred into the prepolymer solutions. Preferably the water is added to the dissolved prepolymer.

The solvent still present in the dispersions after the dispersing step is typically then removed by distillation. Its removal during the actual dispersing is likewise possible.

The solids content of the polyurethane dispersion is between 20% to 70% by weight, preferably 20% to 65% by weight. For coating experiments these dispersions can be diluted arbitrarily with water, in order to allow the thickness of the coating to be varied.

The polyurethane dispersions used in accordance with the invention are then obtained by mixing a nonionically stabilized polyarethaneurea as defined above, and/or a polyurethaneurea as obtained above, with a silver-containing constituent as defined above, it being possible for the mixture to be homogenized by stirring or shaking.

### Production of the Medical Devices of the Invention

The present invention additionally provides a process for producing antimicrobially equipped medical devices, comprising at least the applying of a coating composition as described above to a surface of a medical device, and the subsequent curing to a coating.

The polyurethaneurea dispersions of the invention can be used to coat a variety of substrates such as, for example, metal, plastic, ceramic, paper, leather or textile fabric. The coatings may be applied by a variety of techniques such as spraying, dipping, knifecoating, printing, spincoating or transfer coating, to any conceivable substrates. Preferred applications of these hydrophilic coatings are for surfaces of medical devices and implants, as already mentioned above.

The present invention further provides for the use of a medical device according to any one of claims 1 to 7 or 10 in medical technology.

The advantages of the polyurethaneurea solutions of the invention are set out by means of comparative experiments in the following examples.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLES

The NCO content of the resins described in the inventive and comparative examples was determined by titration in accordance with DIN EN ISO 11909.

The solids contents were determined in accordance with DIN-EN ISO 3251. 1 g of polyurethane dispersion was dried at 115° C. to constant weight (15-20 min) using an infrared drier.

The average particle sizes of the polyurethane dispersions are measured using the High Performance Particle Sizer (HPPS 3.3) from Malvern Instruments.

Unless noted otherwise, amounts indicated in % are % by weight and relate to the overall solution obtained.

Substances and Abbreviations used:

Desmophen C2200: Polycarbonate polyol, OH number 56 mg KOH/g, number-average molecular weight 2000 g/mol (Bayer, AG, Leverkusen, DE)

Desmophen C1200: Polycarbonate polyol, OH number 56 mg KOH/g, number-average molecular weight 2000 g/mol (Bayer, AG, Leverkusen, DE)

PolyTHF® 2000: Polytetramethylene glycol polyol, OH number 56 mg KOH/g, number-average molecular weight 2000 g/mol (BASF AG, Ludwigshafen, DE)

Polyether LB 25: (monofunctional polyether based on ethylene oxide/propylene oxide, number-average molecular weight 2250 g/mol, OH number 25 mg KOH/g (Bayer AG, Leverkusen, DE)

### Example 1

This example describes the preparation of an inventive polyurethaneurea dispersion.

277.2 g of Desmophen C 2200, 33.1 g of Polyether LB 25 and 6.7 g of neopentyl glycol were introduced at 65° C. and homogenized by stirring for 5 min. This mixture was admixed at 65° C. over the course of 1 min first with 71.3 g of 4,4′-bis(isocyanatocyclobexyl)methane (H₁₂MDI) and thereafter with 11.9 g of isophorone diisocyanate. The reaction mixture was heated to 110° C. After 3 h 40 min the theoretical NCO value was reached. The completed prepolymer was dissolved at 50° C. in 711 g of acetone and then at 40° C. a solution of 4.8 g of ethylenediamine in 16 g of water was metered in over the course of 10 min. The subsequent stirring time was 15 min. Thereafter, over the course of 15 min, dispersion was carried out by addition of 590 g of water. This was followed by the removal of the solvent by vacuum distillation. This gave a storage-stable polyurethane dispersion having a solids content of 41.5% and an average particle size of 164 nm.

### Example 2

This example describes the preparation of an inventive polyurethaneurea dispersion.

277.2 g of Desmophen C 1200, 33.1 g of Polyether LB 25 and 6.7 g of neopentyl glycol were introduced at 65° C. and homogenized by stirring for 5 min. This mixture was admixed at 65° C. over the course of 1 min first with 71.3 g of 4,4′-bis(isocyanatocyclobexyl)methane (H₁₂MDI) and thereafter with 11.9 g of isophorone diisocyanate. The reaction mixture was heated to 110° C. After 2.5 h the theoretical NCO value was reached. The completed prepolymer was dissolved at 50° C. in 711 g of acetone and then at 40° C. a solution of 4.8 g of ethylenediamine in 16 g of water was metered in over the course of 10 min. The subsequent stirring time was 5 min. Thereafter, over the course of 15 min, dispersion was carried out by addition of 590 g of water. This was followed by the removal of the solvent by vacuum distillation. This gave a storage-stable polyurethane dispersion having a solids content of 40.4% and an average particle size of 146 nm.

### Example 3

This example describes the preparation of an inventive polyurethaneurea dispersion.

277.2 g of PolyTHF 2000, 33.1 g of Polyether LB 25 and 6.7 g of neopentyl glycol were introduced at 65° C. and homogenized by stirring for 5 min. This mixture was admixed at 65° C. over the course of 1 min first with 71.3 g of 4,4′-bis(isocyanatocyclohexyl)methane (H₁₂MDI) and thereafter with 11.9 g of isophorone diisocyanate. The reaction mixture was heated to 110° C. After 18 h the theoretical NCO value was reached. The completed prepolymer was dissolved at 50° C. in 711 g of acetone and then at 40° C. a solution of 4.8 g of ethylenediamine in 16 g of water was metered in over the course of 10 min. The subsequent stirring time was 5 min. Thereafter, over the course of 15 min, dispersion was carried out by addition of 590 g of water. This was followed by the removal of the solvent by vacuum distillation. This gave a storage-stable polyurethane dispersion having a solids content of 40.7% and an average particle size of 166 nm.

### Example 4

This example describes the preparation of an inventive polyurethaneurea dispersion.

269.8 g of PolyTHF 2000, 49.7 g of Polyether LB 25 and 6.7 g of neopentyl glycol were introduced at 65° C. and homogenized by stirring for 5 min. This mixture was admixed at 65° C. over the course of 1 min first with 71.3 g of 4,4′-bis(isocyanatocyclohexyl)methane (H₁₂MDI) and thereafter with 11.9 g of isophorone diisocyanate. The reaction mixture was heated to 110° C. After 17.5 h the theoretical NCO value was reached. The completed prepolymer was dissolved at 50° C. in 711 g of acetone and then at 40° C. a solution of 4.8 g of ethylenediamine in 16 g of water was metered in over the course of 10 min. The subsequent stirring time was 5 min. Thereafter, over the course of 15 min, dispersion was carried out by addition of 590 g of water. This was followed by the removal of the solvent by vacuum distillation. This gave a storage-stable polyurethane dispersion having a solids content of 41.6% and an average particle size of 107 nm.

### Example 5

This example describes the preparation of an inventive polyurethaneurea dispersion.

282.1 g of PolyTHF 2000, 22.0 g of Polyether LB 25 and 6.7 g of neopentyl glycol were introduced at 65° C. and homogenized by stirring for 5 min. This mixture was admixed at 65° C. over the course of 1 min first with 71.3 g of 4,4′-bis(isocyanatocyclohexyl)methane (H₁₂MDI) and thereafter with 11.9 g of isophorone diisocyanate. The reaction mixture was heated to 110° C. After 21.5 h the theoretical NCO value was reached. The completed prepolymer was dissolved at 50° C. in 711 g of acetone and then at 40° C. a solution of 4.8 g of ethylenediamine in 16 g of water was metered in over the course of 10 min. The subsequent stirring time was 5 min. Thereafter, over the course of 15 min, dispersion was carried out by addition of 590 g of water. This was followed by the removal of the solvent by vacuum distillation. This gave a storage-stable polyurethane dispersion having a solids content of 37.5% and an average particle size of 195 nm.

### Example 6

A 0.054 molar silver nitrate solution was admixed with a mixture of 0.054 molar sodium hydroxide solution and the dispersing assistant Disperbyk 190 (manufacturer: BYK Chemie) (1 g/l) in a volume ratio of 1:1 and the mixture was stirred for 10 min. A brown A_{g2}O nanosol was formed. Added to this reaction mixture with stirring was an aqueous 4.6 molar formaldehyde solution, and so the molar ratio of Ag+ to reducing agent was 1:10. This mixture was heated to 60° C., held at that temperature for 30 min and then cooled. The particles were purified by centrifugation (60 min at 30 000 rpm) and redispersed in fully demineralized water by introduction of ultrasound (1 min). This operation was repeated twice. A colloidally stable sol with a solids content of 5% by weight (silver particles and dispersing assistant) was obtained in this way. The yield is just under 100%. After centrifugation, according to elemental analysis, the silver dispersion contains 3% by weight of Disperbyk 190, based on the silver content. An analysis by means of laser correlation spectroscopy showed an effective diameter for the particles of 73 nm.

50 ml of the polyurethane dispersions from Examples 1 to 5 were admixed with a 15.1% aqueous colloidal silver dispersion, whose preparation is described above, and the mixtures were homogenized by shaking. The amount of silver dispersion added to the polyurethane dispersions of Examples 1 to 5 is such that the dispersions contain 1% by weight of silver, based on the solid polymer content.

TABLE 1
Polyurethane dispersions with 1% by
weight of nanocrystalline silver

| Example | Product |
|---|---|
| 6a | PU dispersions of Example 1 with 1% by weight |
| | nanocrystalline silver |
| 6b | PU dispersions of Example 2 with 1% by weight |
| | nanocrystalline silver |
| 6c | PU dispersions of Example 3 with 1% by weight |
| | nanocrystalline silver |
| 6d | PU dispersions of Example 4 with 1% by weight |
| | nanocrystalline silver |
| 6e | PU dispersions of Example 5 with 1% by weight |
| | nanocrystalline silver |

### Example 7

### Ag Release Study, Chemical

The silver-containing coatings for the measurement of silver release were produced on glass slides measuring 25×75 mm with the aid of a spincoater (RC5 Gyrset 5, Karl Süss, Garching, Germany). For this purpose a slide treated with 3-aminopropyltriethoxysilane to improve adhesion was clamped in on the sample plate of the spincoater and was covered homogeneously with about 2.5-3 g of aqueous undiluted polyurethane dispersion. Rotation of the sample plate at 1300 revolutions per minute for 20 sec gave a homogeneous coating, which was dried at 100° C. for 15 min and then at 50° C. for 24 h. From the slides thus obtained, sections of 4.5 cm2 were produced and were used for measuring the amounts of silver released.

The slide sections with various silver-containing polyurethane coatings of Examples 6a to 6e were covered with 2.5 ml of distilled water in a tablet tube and stored in an incubator at 37° C. for one week. The water was removed and the amount of silver delivered from the film to the liquid was determined by atomic absorption spectroscopy. The dry films on the glass sections were again covered with 2.5 ml of water and stored further at 37° C. The entire process was repeated 5 times, allowing silver release to be determined for a number of weeks.

TABLE 2
Silver release of the films in an aqueous environment

| | | Example | Example | Example | Example |
|---|---|---|---|---|---|
| | Example 6a | 6b | 6c | 6d | 6e |
| ng Ag | 450 | 475 | 1350 | 1475 | 400 |
| (Week 1) | | | | | |
| ng Ag | 77.5 | 400 | 400 | 625 | 182.5 |
| (Week 2) | | | | | |
| ng Ag | 35 | 170 | 247.5 | 500 | 185 |
| (Week 3) | | | | | |
| ng Ag | 12.5 | 82.5 | 230 | 250 | 80 |
| (Week 4) | | | | | |
| ng Ag | 12.5 | 45 | 1025 | 275 | 60 |
| (Week 5) | | | | | |

The results show that the coatings deliver silver over a relatively long period of time.

### Example 8

2.5 g of the silver-containing polyurethane dispersions of Examples 6b and 6d were weighed out into aluminium containers (6.4 cm diameter, 1.3 cm high). The aqueous dispersion was first left to dry at room temperature for 2 h and then the polymers, still moist, were dried in a drying cabinet at 50° C. for 25 h. The resultant polyurethane mouldings were parted from the aluminium trays, and small plaques with a diameter of 5 mm were cut. These silver-containing polyurethane plaques with a thickness of 150 μm were tested for their antimicrobial activity against Escherichia coli.

Polyurethane plaques comprising the silver-containing polyurethane dispersions of Examples 6b and 6d, with a diameter of 5 mm, were investigated for their bactericidal action in a bacterial suspension of Escherichia coli ATCC 25922.

A bacterial culture of Escherichia coli ATCC 25922 was prepared by taking colonies from an agar plate which had been colonized and grown overnight at 37° C., with Columbia Agar (Columbia blood agar plates, Becton Dickinson, # 254071), and suspending the colonies in 0.9% strength sodium chloride solution. An aliquot of this solution was transferred into PBS (PBS pH 7.2, Gibco, #20012) with 5% Müller Hinton medium (Becton Dickinson, #257092), to give an OD600 of 0.0001. This solution corresponds to a microbe count of 1×10⁵ microbes per ml. The microbe count was determined by carrying out serial dilution and plating out the dilution stages on agar plates. The cell count was reported in the form of colony-forming units (CFU/ml).

The polyurethane plaques comprising the silver-containing polyurethane dispersions of Examples 6b and 6d, with a diameter of 5 mm, were each placed in one well of a 24-well microtitre plate. Pipetted into each of the wells, onto the plaques, was 1 ml of the E. coli ATCC 25922 suspension with a microbe count of 1×10⁵ microbes per ml, and the plate was incubated at 37° C. for 24 h. Immediately after preparation of the experiment, and after 2, 4, 6 and 24 h, 20 μl were withdrawn per well and a microbe count determination was carried out. After 24 h, the sample plaques were removed and transferred to a new 24-well microtitre plate. Applied to the plaques, again, was 1 ml of a bacterial suspension of E. coli ATCC 25922, prepared as above, followed by incubation at 37° C.; and, immediately after experimental preparation and after 24 h, 20 μl of each sample were removed, and the microbe count was determined. This was repeated for up to 10 days.

TABLE 3a
Investigation of the antibacterial action of the
polyurethane coating of Example 6b

| | Day 1 | Day 2 | Day 3 | Day 4 | Day 8 |
|---|---|---|---|---|---|
| Microbe | 1.5 × 10⁵ | 1.0 × 10⁵ | 1.0 × 10⁵ | 1.5 × 10⁵ | 1.0 × 10⁵ |
| count/ml | | | | | |
| (0 h) | | | | | |
| Microbe | <100 | 1.6 × 10² | 1.6 × 10² | 3.0 × 10³ | <100 |
| count/ml | | | | | |
| (24 h) | | | | | |

TABLE 3b
Investigation of the antibacterial action of the
polyurethane coating of Example 6d

| | Day 1 | Day 2 | Day 3 | Day 4 | Day 8 | Day 9 | Day 10 |
|---|---|---|---|---|---|---|---|
| Microbe | 1.5 × 10⁵ | 1.0 × 10⁵ | 1.0 × 10⁵ | 1.5 × 10⁵ | 1.0 × 10⁵ | 1.5 × 10⁵ | 2.0 × 10⁵ |
| count/ml | | | | | | | |
| (0 h) | | | | | | | |
| Microbe | <100 | 9 × 10² | 9 × 10² | 1.4 × 10² | <100 | <100 | <100 |
| count/ml | | | | | | | |
| (24 h) | | | | | | | |

The results demonstrate an antibacterial coating of more than one week. Bacterial suspension added freshly is continually killed back down to very low microbe counts.

### Example 9

The silver-containing coatings for the measurement of antimicrobial activity were produced on glass slides measuring 25×75 mm with the aid of a spincoater (RC5 Gyrset 5, Karl Süss, Garching, Germany). For this purpose a slide treated with 3-aminopropyltriethoxysilane for improved adhesion was clamped in on the sample plate of the spincoater and was covered homogeneously with about 2.5-3 g of aqueous undiluted polyurethane dispersion. Rotation of the sample plate at 1300 revolutions per minute for 20 sec gave a homogeneous coating, which was dried at 100° C. for 15 min and then at 50° C. for 24 h. The coated slides obtained were employed directly for the measurement of the antimicrobial activity.

The test of antimicrobial activity was carried out in accordance with the following specification:

The test microbe, E. coli ATCC 25922, was cultured in an overnight culture on Columbia Agar (Columbia blood agar plates, Becton Dickinson, # 254071) at 37° C. Thereafter colonies were suspended in PBS (PBS pH 7.2, Gibco, #20012) with 5% Müller Hinton medium (Becton Dickinson, #257092) and a cell count of approximately 1×10⁵ microbes/ml was set (“microbial suspension”). The test material was transferred to a Falcon tube filled with 30 ml of microbial suspension, and was incubated at 37° C. overnight. After 24 h, 20 μl of the cell suspension were taken for the purpose of monitoring growth. The cell count was determined by serial dilution and by plating-out of the dilution stages onto agar plates. The cell count is reported in the form of colony-forming units (CFU/ml).

TABLE 4a
Antibacterial action of polyurethane coatings

| | Example | | Example | | Example |
|---|---|---|---|---|---|
| | 6a | Example 6b | 6c | Example 6d | 6e |
| CFU/ml | 4 × 10⁵ | 4 × 10⁵ | 4 × 10⁵ | 4 × 10⁵ | 4 × 10⁵ |
| (0 h) | | | | | |
| CFU/ml | <100 | <100 | <100 | <100 | <100 |
| (24 h) | | | | | |

The coating of Example 6d was tested for antimicrobial action over 15 days. For this purpose the above specification was continued in such a way that, following determination of the microbe count, fresh bacterial suspension of known concentration was applied to the coating again, and after 24 h a microbe count is determined again. The film was irrigated in PBS buffer on days 4-6 and 9-14.

TABLE 4b
Long-term action of the antibacterial coating of Example 6d

| | Day 1 | Day 2 | Day 3 | Day 4 | Day 8 | Day 15 |
|---|---|---|---|---|---|---|
| Microbe | 4 × 10⁵ | 7 × 10⁵ | 6 × 10⁵ | 4 × 10⁵ | 3 × 10⁵ | 6 × 10⁵ |
| count/ml | | | | | | |
| (0 h) | | | | | | |
| Microbe | <100 | <100 | <100 | <100 | <100 | <100 |
| count/ml | | | | | | |
| (24 h) | | | | | | |

### Example 10

### Comparative Experiment:

Silver release from sulphonate-containing dispersions

Dispercoll® U 53 and Impranil® DLN are used. Both are polyester-containing polyurethaneureas based on aliphatic diisocyanates and stabilized by sulphonic acid groups

| | Dispercoll U 53 | Impranil DLN |
|---|---|---|
| ng Ag (week 1) | 248 | 325 |
| ng Ag (week 2) | 70 | 98 |
| ng Ag (week 3) | 22 | 122 |
| ng Ag (week 4) | 23 | 20 |
| ng Ag (week 5) | 142 | 525 |
| ng Ag (week 6) | 1100 | 1575 |

Comparison to nonionic dispersions of the invention:
In the first and second weeks there is a relatively low level of silver release in comparison to the nonionic dispersions of the invention.
Both sulphonate-containing coatings become hard and brittle after storage for 4 to 5 weeks. The sharp increase in release of silver in weeks 5 and 6 can be explained by the decomposition of the polymer matrix.

### Example 14

### Comparative Experiment

Furthermore, in studies of the bacterial adherence of E. coli on different polyurethane surfaces it is evident that two nonionic dispersions (Examples 2 and 4) have a relatively low affinity for E. coli even without the use of Ag. The experiment was carried out by a method based on Japanese standard JIS 2801.

For this purpose the test microbe, E. coli ATCC 25922, was cultured in an overnight culture on Columbia agar at 37° C. Subsequently a number of colonies were suspended in phosphate-buffered saline (PBS) with 5% Müller Hinton medium, and a cell count of approximately 1×10⁵ microbes/ml was set. 100 μl of each of these suspensions was distributed over the test material (in this case, the coatings without nanocrystalline silver) using a piece of Parafilm measuring 20×20 mm, so that the surface is wetted uniformly with cell suspension. Subsequently the test material with the bacterial suspension was incubated in a humidity chamber at 37° C. for 6 h. After 6 h, 20 μl of the cell suspension were taken for the purpose of monitoring growth. The cell count was determined by carrying out serial dilution and plating out the dilution stages on agar plates. Only living cells were determined in this case. For all of the materials studied, a growth of the microbes to approximately 10⁷ to 10⁸ microbes/ml was found. The test materials without the addition of nanocrystalline silver therefore do not hinder microbial growth.

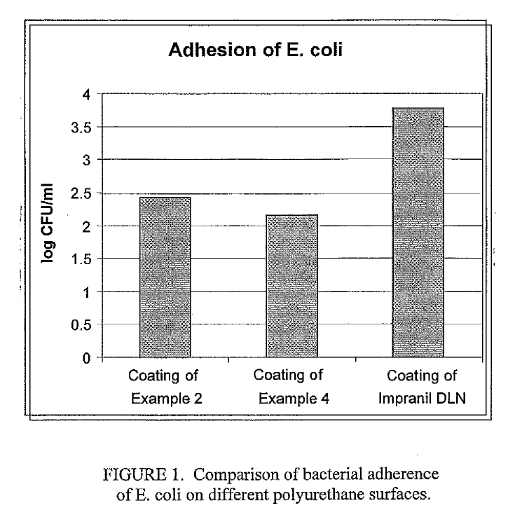

Subsequently the Parafilm was removed from the test material and the test material was washed with three times 4 ml of PBS in order to remove free-floating cells. The test material was then transferred to 15 ml of PBS and sonicated in an ultrasound bath for 30 seconds, in order to detach the bacteria adhering to the surface of the test material. The results are shown in FIG. 1, wherein colony-forming units per ml are expressed as log stages.

From this result it can be inferred that nonionic dispersions exhibit a relatively low affinity for E. coli in comparison to a sulphonate-containing dispersion (Impranil DLN) without further auxiliaries. On its own, however, this still does not protect against infection, but the relatively low concentration of bacteria on the surface can then easily be degraded fully with a relatively low concentration of silver.

1. A medical device comprising a coating obtained from an aqueous dispersion, said aqueous dispersion comprising at least one nonionically stabilized polyurethaneurea and at least one silver-containing constituent.

2. The medical device of claim 1, wherein said at least one nonionically stabilized polyurethaneurea comprises a macropolyol synthesis component selected from the group consisting of polyester polyols, polycarbonate polyols, polyether polyols, and mixtures thereof.

3. The medical device of claim 2, wherein said macropolyol synthesis component is selected from the group consisting of a polyether polyol and a polycarbonate polyol.

4. The medical device of claim 1, wherein the polyurethaneurea of said coating is synthesized from at least the following synthesis components:
a) at least one macropolyol;
b) at least one polyisocyanate;
c) at least one diamine or amino alcohol;
d) at least one monofunctional polyoxyalkylene ether; and
h) antimicrobially active silver.

5. The medical device of claim 1, wherein said at least one silver-containing constituent is a, high-porosity silver powder, silver on support material, or a colloidal silver sol.

6. The medical device of claim 1, wherein said coating comprises nanocrystalline silver particles with an average size in the range of from 1 to 1000 nm are used.

7. The medical device of claim 1, wherein the amount of silver present in said coating, based on the amount of solid nonionically stabilized polyurethaneurea and calculated as Ag and Ag+, is in the range of from 0.1% to 10% by weight.

8. A process for producing a medical device comprising at least one coating, comprising applying an aqueous dispersion comprising at least one nonionically stabilized polyurethaneurea and at least one silver-containing constituent to said medical device.

9. The process of claim 8, comprising applying said aqueous dispersion to said medical device by knifecoating, printing, transfer coating, spraying, spin coating, or dipping.

10. A medical device comprising a coating obtained by the process of claim 8.

11. The medical device of claim 1, wherein said medical device is selected from the group consisting of contact lenses; cannulas; catheters; urological catheters; urinary catheters; ureteral catheters; central venous catheters; venous catheters; inlet catheters; outlet catheters; dilation balloons; catheters for angioplasty; catheters for biopsy; catheters for introducing a stent; catheters for introducing an embolism filter; catheters for introducing a vena cava filter; balloon catheters; expandable medical devices; endoscopes; laryngoscopes; tracheal devices; endotracheal tubes; respirators; tracheal aspiration devices; bronchoalveolar lavage catheters; catheters used in coronary angioplasty; guide rods; insertion guides; vascular plugs; pacemaker components; cochlear implants; dental implant tubes for feeding; drainage tubes; guide wires; gloves; stents; implants; extracorporeal blood lines; membranes, dialysis membranes; blood filters; devices for circulatory support; dressing materials for wound management; urine bags; stoma bags; implants which comprise a medically active agent; stents which comprise a medically active agent; balloon surfaces which comprise a medically active agent; contraceptives which comprise a medically active agent; endoscopes; laryngoscopes; and feeding tubes.
